(19) United States
(12) Patent Application Publication
(10) Pub. No.: US 2026/0264692 A1
(43) Pub. Date: Sep. 10, 2026

(54) SYSTEM AND METHOD FOR MONITORING HEALTH OF ONE OR MORE USERS OF A TWO-WHEELED VEHICLE

(71) Applicant: TVS MOTOR COMPANY LIMITED, Chennai (IN)

(72) Inventors: Satakshi Roy, Chennai (IN); Ajay Kumar Vasu, Chennai (IN); Datta Rajaram Sagare, Chennai (IN)

(73) Assignee: TVS MOTOR COMPANY LIMITED, Chennai (IN)

(21) Appl. No.: 18/876,428

(22) PCT Filed: Mar. 7, 2023
(86) PCT No.: PCT/IN2023/050217

§ 371 Date: Dec. 18, 2024

(30) Foreign Application Priority Data
Jun. 23, 2022 (IN) 202241036003

(51) Int. Cl. B60W 40/08; B60W 50/14; B62J 45/416; B62J 50/22

(52) U.S. Cl. CPC B60W 40/08; B60W 50/14; B62J 45/416; B62J 50/22; B60W 2040/0872; B60W 2050/146; B60W 2300/36; B60W 2540/221

(57) ABSTRACT

A system, for monitoring health of one or more users of a two-wheeled vehicle, includes: at least one sensor disposed at one or more locations on the vehicle, each of the at least one sensor configured to generate a heartrate detection signal upon contact with each of the one or more users; and a control unit disposed in the vehicle and communicably coupled to each of the at least one sensor. The control unit configured to: receive, the heartrate detection signal from the at least one sensor; compute, health parameters corresponding to each of the one or more users based on the heartrate detection signal; compare, the computed health parameters with reference health parameters; and alert, the one or more users prior to starting of the vehicle, when at least one computed health parameter deviates from the corresponding reference health parameter.

### FIELD OF THE INVENTION

The present invention relates to a method and a system for monitoring health of one or more users of a vehicle. More particularly, relates to the method and system for monitoring of one or more users of a two-wheeled vehicle.

### BACKGROUND OF THE INVENTION

It is a known fact that a rider health monitoring system in a two-wheeler vehicle is not pre-dominant. It is essential, as nearly 1.3 million people succumb to road crashes each year. An additional 20-50 million are injured or disabled. As such, road safety for riders of the two-wheeled vehicles is of utmost importance.

One of the major reasons for accidents or crashes in roads is due to fatigue among drivers. Fatigue is typically defined as a gradual and cumulative process associated with ‘a loss of efficiency, and a disinclination for any kind of effort’. Fatigue increases as time-on-task or the length of time of riding of the two-wheeled vehicle progresses. It is estimated that nearly 20% of all accidents is caused due to fatigue as per The Royal society for prevention of accidents (RoSPA). The road accidents which involve two wheelers have the highest share of 33.9% in total accidents and of 29.8% in total fatalities. Development of Rider assistance systems is therefore, of utmost importance to improve rider experience in terms of comfort and safety.

To overcome the aforementioned problems, rider health monitoring systems for vehicles have been developed. These systems are adapted to monitor condition or state of a driver of the vehicle. However, these systems typically do not alert the rider before start of the rider, which is critical in certain scenarios. Also, these systems do not monitor heart-rate of the rider which is again crucial in certain scenarios. Furthermore, these systems monitor only health of the riders and thus do not consider monitoring health of co-passengers or pillion rider's health, which is undesirable.

In view of the above, there is a need for a system and a method for monitoring health of one or more users of a two-wheeled vehicle, which addresses one or more limitations stated above.

### SUMMARY OF THE INVENTION

In one aspect, a system for monitoring health of one or more users of a two-wheeled vehicle is provided. The system comprises at least one sensor disposed at one or more locations on the vehicle. Each of the at least one sensor is configured to generate a heartrate detection signal upon contact with each of the one or more users, wherein heartrate detection signal is indicative of a heart rate of the corresponding one or more users. A control unit is disposed in the vehicle and communicably coupled to each of the at least one sensor. The control unit is configured to receive, the heartrate detection signal from the at least one sensor, compute health parameters corresponding to each of the one or more users based on the heartrate detection signal, compare, the computed health parameters with reference health parameters and alert, the one or more users prior to starting of the vehicle, when at least one computed health parameter deviates from the corresponding reference health parameter.

In an embodiment, the health parameters computed by the control unit comprises at least one of: Spo2 levels, heart rate variability, maximum heart rate, target heart rate, Body Mass Index (BMI), Tachycardia, Bradycardia, average Inter-beat-intervals and the root mean square of successive differences between normal heartbeats (RMSSD).

In an embodiment, the control unit is communicably coupled to a memory unit for storing data pertaining to the reference health parameters and the computed health parameters.

In an embodiment, the control unit is configured to alert the one or more users via at least one of a visual alert, a tactile alert and an audible alert.

In an embodiment, the control unit is communicably coupled to an instrument cluster of the vehicle. The instrument cluster being capable of alerting the one or more users.

In an embodiment, the instrument cluster comprises a display unit configured to one of display, the health parameters corresponding to the one or more users upon computation by the control unit and visually alert, the one or more users when at least one computed health parameter deviates from the corresponding reference health parameter.

In an embodiment, the at least one sensor and the control unit are communicably coupled to a battery module of the vehicle.

In an embodiment, the system is capable of being activated via one of: a switch mounted on a handlebar of the vehicle, a voice command from the one or more users, and by contacting the at least one sensor via a fingertip of the one or more users.

In an embodiment, the control unit is adapted to disconnect an ignition system of the vehicle from a battery module for preventing starting of the vehicle by the one or more users, when at least one computed health parameter deviates from the corresponding reference health parameter.

In an embodiment, the at least one sensor is disposed on a handlebar for monitoring health parameters of a rider of the vehicle or on a seat for monitoring health parameters of a pillion rider of the vehicle.

In an embodiment, each of the at least one sensor is a fingerprint sensor.

In an embodiment, the control unit is configured to monitor the health parameters of the one or more user periodically for a predetermined duration of time.

In another aspect, a method of monitoring health of one or more users of a two-wheeled vehicle is provided. The method comprises receiving by the control unit a heartrate detection signal from at least one sensor, the at least one sensor being disposed at one or more locations on the vehicle, wherein each of the at least one sensor is configured to generate the heartrate detection signal upon contact with each of the one or more users, the heartrate detection signal being indicative of a heart rate of the corresponding one or more users. The control unit then computes health parameters corresponding to each of the one or more users based on the heartrate detection signal. The computed health parameters are thereafter compared by the control unit with reference health parameters. Subsequently, the control unit alerts the one or more users prior to starting of the vehicle, when at least one computed health parameter deviates from the corresponding reference health parameter.

### BRIEF DESCRIPTION OF THE DRAWINGS

Reference will be made to embodiments of the invention, examples of which may be illustrated in accompanying figures. These figures are intended to be illustrative, not limiting. Although the invention is generally described in context of these embodiments, it should be understood that it is not intended to limit the scope of the invention to these particular embodiments.

FIG. 1 is a schematic view of a vehicle, in accordance with an embodiment of the present invention.

FIG. 2 is a schematic view of a system for monitoring health of one or more users of the vehicle, in accordance with an embodiment of the present invention.

FIG. 3 is a top view of a handle of the vehicle, in accordance with an embodiment of the present invention.

FIG. 4 is a top view of a seat of the vehicle, in accordance with an embodiment of the present invention.

FIG. 5 is a schematic view of an instrument cluster of the vehicle, in accordance with an embodiment of the present invention.

FIG. 6 is a graphical representation of a waveform of a heartrate of a user, in accordance with an embodiment of the present invention.

FIG. 7 is a flow diagram of a method for monitoring health of one or more users of the vehicle, in accordance with an embodiment of the present invention.

### DETAILED DESCRIPTION OF THE INVENTION

Various features and embodiments of the present invention here will be discernible from the following further description thereof, set out hereunder. In the ensuing exemplary embodiments, the vehicle can be a two-wheeled vehicle.

The present invention relates to a system and a method for monitoring health of one or more users of a vehicle. Particularly, the present invention relates to the method and the system for monitoring health of one or more users of a two-wheeled vehicle.

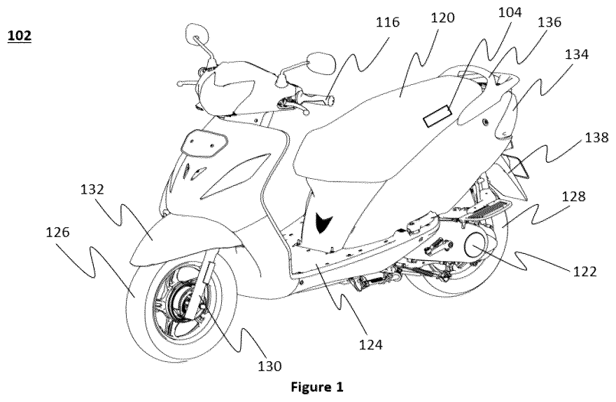

FIG. 1 illustrates a schematic view of a vehicle 102, in accordance with an embodiment of the present invention. As an example, the vehicle 102 is a scooter-type vehicle or a motorcycle. The vehicle 102 has a powertrain component 122, which can be a prime mover that is adapted to generate motive force required for movement of the vehicle 102. In an embodiment, the powertrain component 122 is an internal combustion engine or an electric motor of the vehicle 102. In another embodiment, the powertrain component 122 is a combination of the prime mover and a transmission system (not shown), that is disposed behind a floorboard 124 and below a seat 120 and/or a storage bin (not shown). The vehicle 200 has a front wheel 126, a rear wheel 128 and a frame member (not shown in Figures).

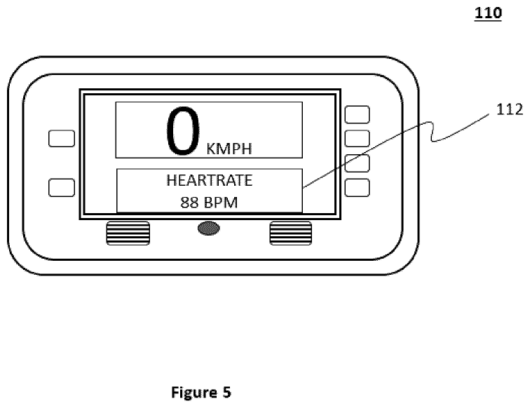

The frame member comprises a head pipe (not shown in Figures) that is adapted to support a steering shaft (not shown) and a front suspension 130 attached to the steering shaft through a lower bracket (not shown). The front suspension 130 supports the front wheel 126. The upper portion of the front wheel 126 is covered by a front fender 132 mounted to the front suspension 130. In an embodiment, the front fender 132 is movable along with the front wheel 126, during travel over undulations on a road surface. A handlebar 116 is fixed to upper bracket (not shown) and can rotate about the steering shaft for turning the vehicle 102. A headlight (not shown) and an instrument cluster 110 (for e.g. shown in FIG. 5) is arranged on an upper portion of the head pipe. In an embodiment, the instrument cluster 110 is a digital instrument or an analog instrument or a combination thereof. The instrument cluster 110 may be provided with gauges such as speedometer (not shown), tachometer (not shown), fuel gauge (not shown) and the like, as per design feasibility and requirement. In an embodiment, the instrument cluster 100 is provided with switches (not referenced in Figures) capable of receiving data pertaining to the user such as, age of the user, weight of the user, height of the user and the like.

Further, a shock absorber assembly (not shown) is provided to the rear wheel 128 for dampening the vibrations induced during travel of the vehicle 102 over undulations on the road surface. In an embodiment, the powertrain component 122 has one end mounted to the frame member and an other end mounted to the shock absorber assembly. As such, the powertrain component 122 is suspended on the other end via the shock absorber assembly. A taillight unit 134 is disposed at the end of the vehicle 102 and at the rear of the seat 120. A grab rail 136 is also provided for facilitating the grip and/or balance to one or more users on the vehicle 102 during movement. In an embodiment, the one or more users (not shown) pertains to a rider of the vehicle 102 and/or a pillion rider of the vehicle 102. In the present description, for brevity the one or more users is referred collectively as a user. The rear wheel 128 is arranged below the seat 120 and adapted to receive the motive force from the powertrain component 122. The transmission assembly is provided for transferring the motive force from the prime mover onto the rear wheel 128 for driving the vehicle 102. In an embodiment, the transmission assembly may include an endless transmission drive such as a chain drive or a belt drive, for transferring the motive force to the rear wheel 128. A rear fender 138 is disposed above the rear wheel 128.

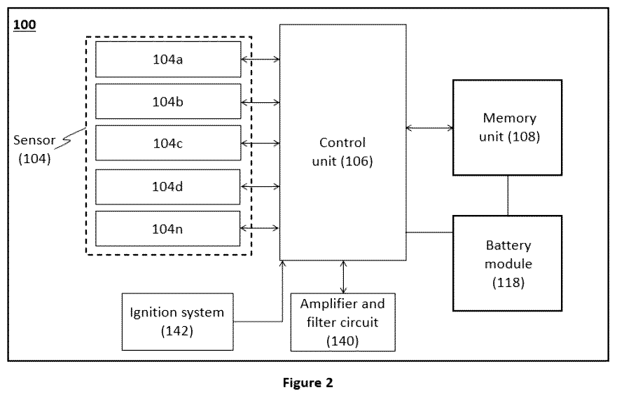

Referring to FIG. 2 in conjunction with FIG. 1, the vehicle 102 comprises the system 100 for monitoring health of the one or more users. The system 100 is adapted to monitor health of the rider and/or the pillion rider or co-passenger of the vehicle 102, thereby ensuring safety.

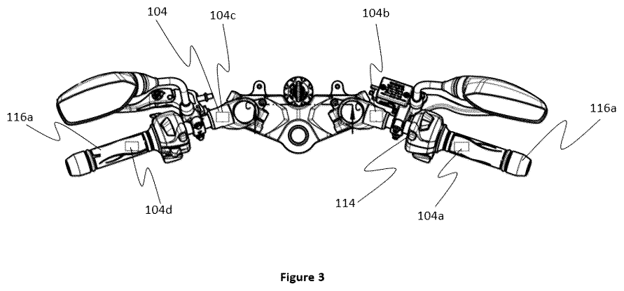

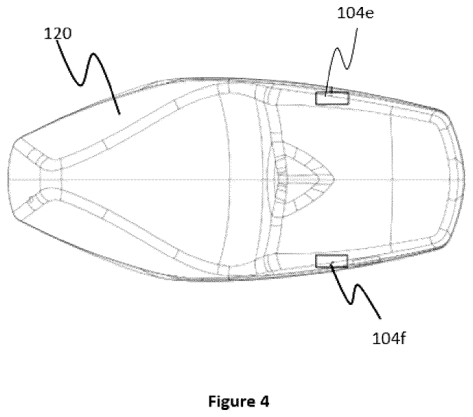

The system 100 comprises at least one sensor 104 disposed strategically at one or more locations on the vehicle 102. Each of the at least one sensor 104 are configured to generate a heartrate detection signal upon contact with each of the one or more users, wherein the heartrate detection signal is indicative of a heartrate of the corresponding one or more users. In an embodiment, each of the sensors 104 is a fingerprint sensor configured to detect heartrate of the one or more users and generate a corresponding heartrate detection signal. In the present embodiment, six sensors are provided on the vehicle 102 for monitoring heartrate of the one or more users, wherein four of the sensors 104a, 104b, 104c and 104d (as shown in FIG. 3) are provided on the handlebar 116, while the other two sensors 104e, 104f (as shown in FIG. 4) are provided on side surfaces of the seat 120 of the vehicle. The sensors 104a, 104d are located on handle grip portions 116a (shown in FIG. 3) of the handlebar 116, while the sensors 104b, 104c are positioned proximal to the steering shaft. Also, the sensors 104e, 104f are provided on side surfaces of the seat 120 of the vehicle 102. In another embodiment, the dimensions of the sensors 104 are selected as per design feasibility and requirement.

The system 100 further includes a control unit 106 disposed in the vehicle 102 and communicably coupled to each of the sensors 104. The control unit 106 is communicably coupled to the one or more sensors 104 via a wired connection or a wireless connection as per design feasibility and requirement. The control unit 106 is adapted to monitor health of the one or more users, based on the heartrate detection signal received from the sensors 104.

In an embodiment, the control unit 106 is also communicably coupled to an ignition system 142 of the vehicle 102. As such, the control unit 106 is capable of controlling operation of the ignition system 142 between an ignition ON condition and an ignition OFF condition of the vehicle 102. As such, the control unit 106 is capable of controlling the ignition condition of the vehicle 102. In an embodiment, the control unit 106 is configured to switch-OFF or disconnect the ignition system 142, when at least one health parameters of the one or more users deviates from a reference parameter. In an embodiment, the control unit 106 is adapted to prevent actuation of the ignition ON condition of the vehicle 102, unless health of the one or more users are in accordance with reference parameter. As such, unless the one or more users are healthy, the control unit 106 prevents use of the vehicle 102 by the one or more users.

In an embodiment, the control unit 106 can be configured within an Engine Control Unit (ECU) (not shown) of the vehicle 102. In another embodiment, the control unit 106 can be configured as a separate module mounted with the instrument cluster 110, which can be in communication with the ECU of the vehicle 102. In some embodiments, the control unit 106 may comprise one or more additional components such as, but not limited to, an input/output module, a pre-processing module and an analytic module. In another embodiment, the vehicle 102 may comprise more than one of same or similar control unit(s) 106.

The control unit 106 is in communication with the components such as the processing module (not shown) and the analytic module (not shown). In another embodiment, the control unit 106 may be embodied as a multi-core processor, a single core processor, or a combination of one or more multi-core processors and one or more single core processors. For example, the control unit 106 is embodied as one or more of various processing devices or modules, such as a coprocessor, a microprocessor, a controller, a digital signal processor (DSP), a processing circuitry with or without an accompanying DSP, or various other processing devices including integrated circuits such as, for example, an application specific integrated circuit (ASIC), a field programmable gate array (FPGA), a microcontroller unit (MCU), a hardware accelerator, a special-purpose computer chip, or the like. In yet another embodiment, the control unit 106 may be configured to execute hard-coded functionality. In still another embodiment, the control unit 106 may be embodied as an executor of instructions, where the instructions are specifically configured to the control unit 106 to perform the steps or operations described herein for monitoring health of the one or more users.

Further, the control unit 106 is communicably coupled to a memory unit 108. The memory 108 is capable of storing information processed by the control unit 106 and also the data received from each of the sensors 104. As such, the data received and processed by the control unit 106 is available during a requirement. The memory 108 is embodied as one or more volatile memory devices, one or more non-volatile memory devices and/or combination thereof, such as magnetic storage devices, optical-magnetic storage devices and the like as per design feasibility and requirement. The memory 108 communicates with the control unit 106 via suitable interfaces such as Advanced Technology Attachment (ATA) adapter, a Serial ATA [SATA] adapter, a Small Computer System Interface [SCSI] adapter, a network adapter or any other component enabling communication between the memory 104 and the control unit 106.

In an embodiment, the control unit 106 is also communicably coupled to an amplifier and filter circuit 140, known in the art for filtering and amplifying the heartrate detection signal received from each of the sensors 104. The amplifier and filter circuit 140 are communicably coupled to a battery module 118 for receiving power for filtering and amplifying the heartrate detection signal from the sensors 104.

In an embodiment, the control unit 106 is configured to initiate monitoring of health of the one or more users manually, upon actuation of a switch 114 (shown in FIG. 3) mounted on the handlebar 116 by the one or more users. Alternatively, the control unit 106 is capable of automatically initiate monitoring of the health of the one or more users, upon contact of a finger of the user on the sensor 104. In an embodiment, the control unit 106 is configured to monitor the health parameters of the one or more users periodically for a predetermined duration of time. In the present embodiment, the control unit 106 monitors the health parameters of the one or more users from about 60 seconds to about 2.5 minutes.

The control unit 106 or the analytic module is adapted to monitor the health of the one or more users, based on the heartrate detection signal provided by the sensors 104. In an embodiment, the control unit 106 or the analytic module is adapted to measure health parameters of the one or more users using the heartrate detection signal from photoplethysmography. In an embodiment, the health parameters computed by the control unit 106 comprises one of: Spo2 levels, heart rate variability, maximum heart rate, target heart rate, Body Mass Index (BMI), Tachycardia, Bradycardia, average Inter-beat-intervals and the root mean square of successive differences between normal heartbeats (RMSSD).

Further, the control unit 106 is configured to alert the one or more users when at least one health parameter is deviated from the reference parameters, stored in the memory unit 108. In an embodiment, the control unit 106 is configured to alert the users via at least one of a visual alert, an audible alert and a tactile alert.

In an embodiment, the control unit 106 is configured to provide the visual alert via the display device 112 of the instrument cluster. The visual alert may be provided by indicating the health parameter that is in excess upon comparison with the on the display unit 112.

In an embodiment, the control unit 106 is configured to provide the audible alert via a speaker system (not shown in Figures) disposed on the vehicle 102. In an embodiment, the audible alert may be in the form of an audible tone or a phrase, as per feasibility and requirement. In an embodiment, the speaker system may be integrated within the instrument cluster 110.

In an embodiment, the control unit 106 is configured to provide the tactile alert via a haptic feedback system (not shown) mounted at touchpoints of the vehicle 102. The haptic feedback system is configured to vibrate, thereby providing tactile feedback to the user of the vehicle 102. In an embodiment, the haptic feedback system includes haptic feedback units mounted on handgrip portions 116a or below the seat 120 of the vehicle 120.

In an embodiment, the control unit 106 is configured to display the heartrate determined on a display unit 112 (shown in FIG. 5) provided in the instrument cluster 110 of the vehicle 102.

In an embodiment, when the heartrate determined by the control unit 106 for the user is equal to or greater than 125 Beats Per Minute (BPM), the user is in a state of anxiety or he is performing an excited riding. Such a scenario is alerted to the user by the control unit 106, for indicating present health condition of the user. In another embodiment, when the heartrate determined by the control unit 106 is equal to or below 80 BPM, the user is in a state of drowsiness or fatigue. Such a scenario is also altered by the control unit 106, for indicating sleepiness or fatigued health condition of the user.

In an embodiment, the control unit 106 is configured to compute the Inter-Beat Intervals (IBI), in order to determine abnormalities in heart of the user. The control unit 106 computes the inter-beat intervals between nth and (n−1)th beat as follows:

$$Avg . IBI = \frac{60000}{\text{Beats/} min} \qquad ( eq . 1 )$$

Based on eq. 1, the control unit 106 determines the inter-beat intervals for each heartbeat value computed by the control unit 106.

In an embodiment, the control unit 106 is configured to determine Heart Rate Variability (HRV) of the user. Heart rate variability is a physiological phenomenon of the variation in the time interval between consecutive heartbeats in milliseconds, wherein users with low HRV can easily experience acute stress while users with high HRV rarely experience stress and their cardiovascular system is in great shape. Heart rate variability time-domain indices quantify the amount of HRV observed during monitoring periods that may range from 1 minute to about 24 hours. In the present embodiment, HRV analysis for 2.5 minutes has been considered. Also, in the present embodiment, Root Mean Square of Successive Differences (RMSSD) method is considered for computing the HRV. The RMSSD is obtained by first calculating each successive time difference between heartbeats in milli seconds and then, each of the values are squared and the result is averaged before the square root of the total is obtained, as mentioned below:

$$RMSSD = \sqrt{\frac{1}{N} {\sum }_{i = 1}^{N} ( {\vert {IBI}_{t - 1} - {IBI}_{t} \vert}^{2} )} \qquad ( eq . 2 )$$

In an embodiment, Beats-per-minutes fluctuations observe the fluctuations in our beats per minute in terms of percentage, which is expressed as below:

$$\frac{\vert {BPM}_{n} - {BPM}_{n - 1} \vert}{{BPM}_{n - 1}} \times 100 \% \qquad ( eq . 3 )$$

The control unit 106 is configured to determine the blood oxygen level of the user in percentage as expressed in eq. 3. The normal range for the blood oxygen level is 95% to 100% which ensures that the user is healthy. Values lesser than 92% indicate hypoxemia.

In an embodiment, the display unit 112 is also configured to depict maximum heartrate and a target heartrate for the users, based on age of the users. The maximum heartrate is the highest HR that the user can sustain, while the target heartrate is defined as the minimum number of heartbeats in a given amount of time in order to reach the level of exertion necessary for cardiovascular fitness, specific to a person's age, gender, or physical fitness. In an embodiment, the target heart rate is 50 to 85 percent of the maximum heart rate. The control unit 106 is configured to compute the maximum heart rate as follows:

$$Maximum heartrate = 220 - age \qquad ( eq . 4 )$$

In an embodiment, the control unit 106 computes the target heartrate for users who are intending to visit a gymnasium for a workout, for indicating the maximum heartrate at which the user is required to perform the workout. In an embodiment, the control unit 106 may be communicably coupled to a user device (not shown) such a fitness tracker or a smartphone that is adapted to monitor heartrate of the user. As such, the control unit 106 may obtain the heartrate of the user during the workout through the user device. Based on the heartrate of the user during the workout, the control unit 106 is configured to determine whether the user is fit enough for riding the vehicle 102.

As an example, if the age of the user riding the vehicle 102 is 30, then the maximum heartrate is 220−30=190. Accordingly, the target heartrate of the user is 104 to 160 BPM. In an embodiment, the instrument cluster 110 is capable of receiving data pertaining to age, height, weight and/or any other data of the user. In an embodiment, the instrument cluster 110 receives data pertaining to the user through the fitness tracker or the smartphone or may be manually included by the user.

In an embodiment, the control unit 106 determines heartrate of the one or more users via the sensor 104 which includes an infrared (IR) LED (not shown) and a photodetector diode (not shown) positioned to face each other. When a fingertip of the user is plugged into the sensor 104, the IR LED illuminates the fingertip. The photodetector diode receives the transmitted light through fingertip tissue, and the light is transmitted to the photodetector diode depending on tissue blood volume. As such, intensity of the transmitted light varies with the pulsing of the blood with heartbeat. A plot for the variation in the intensity of the transmitted light is referred as the photoplethysmographic (PPG) signal. The PPG signal transmitted from the photodetector is weak and noisy, which is thereafter modulated through the amplifier and filter circuit (140).

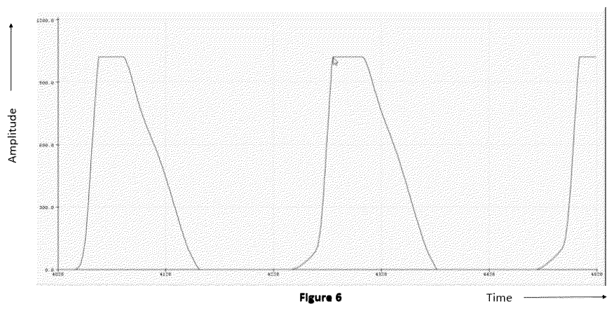

Subsequently, a graphical representation (as shown in FIG. 6) with number of samples of heartbeat on X-axis and Analog to Digital Converted (ADC) values of the intensity or amplitude of the PPG signal along Y-axis is considered by the control unit 106. The Y axis values decides heartbeat based on the intensity of the ADC values. In the present embodiment, a threshold value of 600 is considered on the Y-axis. As such, if the ADC values observed on Y axis in graph is greater than 600, then it is considered as one heartbeat and likewise the values are considered for 15 seconds. The number of heartbeats obtained in 15 seconds is then multiplied by 4 to obtain beats per minute. The interval is considered as 15 seconds, so that the user need not wait for a longer period of time. Additionally, the combination of 15 seconds provides highly accurate heartbeat.

Based on the aforesaid example, if the number of beats determined by the control unit 106 for 15 seconds is 16, then the value is multiplied by 4 to obtain the BPM. That is, 16*4=64 BPM. Further, interbeat intervals (IBI) are determined by considering time taken between two successive heats. The time taken is considered in milliseconds. In an embodiment, the control unit 106 is provided with modules for providing time lapse of values in milliseconds. The time lapse values between two successive heart beats are as presented below:

Interbeat Intervals: 573
10
Interbeat Intervals: 657
11
Interbeat Intervals: 609
12
Interbeat Intervals: 627
13
Interbeat Intervals: 595
14
Interbeat Intervals: 619
15
Interbeat Intervals: 622
16
Interbeat Intervals: 605
17
Interbeat Intervals: 608
18
Interbest Intervals: 627
19
Interbeat Intervals: 615
20
Interbeat Intervals: 603
21
Interbest Intervals: 1244
22
Interbeat Intervals: 653
23
Interbeat Intervals: 602
Beats per Minute: 92
1
Interbeat Intervals: 642
2
Interbeat Intervals: 652

Upon obtaining the time lapse of interbeat intervals, the average interbeat interval is determined using eq. (1). The average interbeat interval is compared with one another to estimate heartbeat rhythm of the user. As an example, if the time taken for a beat (t1) is 600 milliseconds and the time taken for the next immediate beat (t2) is 1200 milliseconds, the time intervals are subtracted. That is, t1−t2=1200−600=600 milliseconds.

Subsequently, the heat rate variability using eq. (2) is computed, wherein it is noted that 88002 is the square of difference of interbeat intervals for a time of 2.5 minutes and 90 is the N value, which is count for number of times the difference of interbeat intervals is computed for 2.5 minutes. Thus, the heartrate variability is computed as square root of (88002/90)=31. Accordingly, when the computed heartrate variability deviates from a nominal range, the user is alerted.

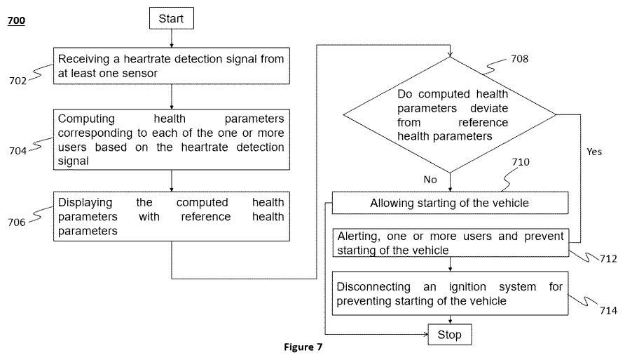

FIG. 7 in one embodiment of the present invention provides a method 700 for determining health parameters of the user.

At step 702, the control unit 106 receives the heartrate detection signal from the sensors 104. The heartrate detection signal may be filtered or amplified via the amplifier and filter unit 140 (as shown in FIG. 2), for filtering noise and amplifying the signal. In an embodiment, the heartrate detection signal generated by the sensors 104 may be from the rider and/or the pillion rider of the vehicle 102. In another embodiment, the data pertaining to heartrate of the rider is obtained by the sensors 104a-104d, while the sensors 104e, 104f obtain the data pertaining to the pillion rider. Upon obtaining the heartrate detection signal from the sensors 104, the control unit 106 proceeds to step 704.

At step 704, the control unit 106 computes the health parameters corresponding to each of the users (i.e. rider and/or the pillion) based on the received heartrate detection signal, as already mentioned above.

At step 706, the control unit 106 displays the computed health parameters in the display unit 112. In an embodiment, the control unit 106 displays the computed health parameters along with the reference health parameters in the display unit 112.

At step 708, the control unit 106 compares the computed health parameter with the reference parameter. As an example, if the computed health parameter is heartrate, and it is determined to be 100, the control unit 106 compares the heartrate of 106 BPM with the reference heartrate which is between 80 BPM and 125 BPM. Since, the heartrate is between the normal range, the control unit 102 proceeds to step 710, for allowing starting of the vehicle 102. In an embodiment, the control unit 106 retains connection between the ignition system 142 and the battery module 118 for enabling starting of the vehicle 102.

When the heartrate detected by the control unit 106 is 130 BPM, the control unit 106 determines that the computed heartrate is deviating from the reference heartrate. In such a scenario, the method 700 moves to step 712 for alerting the one or more users and prevent starting of the vehicle 102. Upon alerting, the method 700 moves to step 714 for disconnecting the ignition system 142 for preventing starting of the vehicle 102. Thus, the system 100 prevents starting or use of the vehicle 102, when the one or more users are unhealthy.

The claimed invention as disclosed above is not routine, conventional or well understood in the art, as the claimed aspects enable the following solutions to the existing problems in conventional technologies. Specifically, the claimed aspect of the monitoring health parameters of the one or more users ensures that safety of rider and/or pillion rider are ensured before starting of the vehicle. Further, due to wireless connectivity of the system, the system is portable. Moreover, the system is configured to monitor health parameters of the user even during a physical activity of the user. Thus, the system is configured to proactively monitor health of the user.

1.-20. (canceled)

21. A system for monitoring health of one or more users of a two-wheeled vehicle, the system comprising:
at least one sensor disposed at one or more locations on the vehicle, each of the at least one sensor configured to generate a heartrate detection signal upon contact with each of the one or more users, wherein the heartrate detection signal is indicative of a heart rate of the corresponding one or more users; and
a control unit disposed in the vehicle and communicably coupled to each of the at least one sensor, the control unit configured to:
receive, the heartrate detection signal from the at least one sensor;
compute, health parameters corresponding to each of the one or more users based on the heartrate detection signal;
compare, the computed health parameters with reference health parameters; and
alert, the one or more users prior to starting of the vehicle, when at least one computed health parameter deviates from the corresponding reference health parameter.

22. The system as claimed in claim 21, wherein
the health parameters computed by the control unit comprises at least one of: Spo2 levels, heart rate variability, maximum heart rate, target heart rate, Body Mass Index (BMI), Tachycardia, Bradycardia, average Inter-beat-intervals and a root mean square of successive differences between normal heartbeats (RMSSD).

23. The system as claimed in claim 21, wherein
the control unit is communicably coupled to a memory unit for storing data pertaining to the reference health parameters and the computed health parameters.

24. The system as claimed in claim 21, wherein
the control unit is configured to alert the one or more users via at least one of a visual alert, a tactile alert and an audible alert.

25. The system as claimed in claim 21, wherein
the control unit is communicably coupled to an instrument cluster of the vehicle, the instrument cluster being capable of alerting the one or more users, and the instrument cluster comprises a display unit configured to one of:
display, the health parameters corresponding to the one or more users upon computation by the control unit; and
visually alert, the one or more users when at least one computed health parameter deviates from the corresponding reference health parameter.

26. The system as claimed in claim 21, wherein
the at least one sensor and the control unit are communicably coupled to a battery module of the vehicle.

27. The system as claimed in claim 21, wherein
the system is capable of being activated via one of: a switch mounted on a handlebar of the vehicle, a voice command from the one or more users, and by contacting the at least one sensor via a fingertip of the one or more users.

28. The system as claimed in claim 21, wherein
the control unit is adapted to disconnect an ignition system of the vehicle from a battery module for preventing starting of the vehicle by the one or more users, when at least one computed health parameter deviates from the corresponding reference health parameter.

29. The system as claimed in claim 21, wherein
the at least one sensor is disposed on a handlebar for monitoring health parameters of a rider of the vehicle or on a seat for monitoring health parameters of a pillion rider of the vehicle.

30. The system as claimed in claim 21, wherein
the control unit is configured to monitor the health parameters of the one or more user periodically for a predetermined duration of time.

31. A method of monitoring health of one or more users of a two-wheeled vehicle, comprising:
receiving, by a control unit, a heartrate detection signal from at least one sensor, the at least one sensor being disposed at one or more locations on the vehicle, wherein each of the at least one sensor is configured to generate the heartrate detection signal upon contact with each of the one or more users, the heartrate detection signal being indicative of a heart rate of the corresponding one or more users;
computing, by the control unit, health parameters corresponding to each of the one or more users based on the heartrate detection signal;
comparing, by the control unit, the computed health parameters with reference health parameters; and
alerting, by the control unit, the one or more users prior to starting of the vehicle, when at least one computed health parameter deviates from the corresponding reference health parameter.

32. The method as claimed in claim 31 comprising,
alerting, by the control unit via an instrument cluster of the vehicle the one or more users, when at least one computed health parameter deviates from the corresponding reference health parameter.

33. The method as claimed in claim 32 comprising,
displaying, by the control unit via a display unit of the instrument cluster one of:
the health parameters corresponding to the one or more users upon computation by the control unit; and
the one or more users when at least one computed health parameter deviates from the corresponding reference health parameter.

34. The method as claimed in claim 31 comprising,
activating, by the control unit, a system for monitoring the health of the one or more users via one of: a switch mounted on a handlebar of the vehicle, a voice command from the one or more users and by contacting the at least one sensor via a fingertip of the one or more users.

35. The method as claimed in claim 31 comprising,
disconnecting, by the control unit, an ignition system of the vehicle from a battery module for prevent starting of the vehicle by the one or more users, when at least one computed health parameter deviates from the corresponding reference health parameter.
